(19) United States
(12) Patent Application Publication
(10) Pub. No.: US 2017/0199194 A1
(43) Pub. Date: Jul. 13, 2017

(54) MULTI-PHENOTYPIC SUBTYPING OF BIOLOGICAL SAMPLES USING SEQUENTIAL FLUORESCENT QUENCHING AND RESTAINING

(71) Applicant: Creatv MicroTech, Inc., Potomac, MD (US)

(72) Inventors: Daniel Adams, Monmouth Junction, NJ (US); Cha-Mei Tang, Potomac, MD (US)

(21) Appl. No.: 15/400,962

(22) Filed: Jan. 6, 2017

Related U.S. Application Data
(60) Provisional application No. 62/374,456, filed on Aug. 12, 2016.
(60) Provisional application No. 62/373,867, filed on Aug. 11, 2016.
(60) Provisional application No. 62/303,243, filed on Mar. 3, 2016.
(60) Provisional application No. 62/275,949, filed on Jan. 7, 2016.

(51) Int. Cl. G01N 33/574; G01N 33/50; G01N 33/58

(52) U.S. Cl. CPC G01N 33/574; G01N 33/582; G01N 33/5091

(57) ABSTRACT

A simple and accurate method for characterizing biomarkers in a biological sample using multiple rounds of fluorescent staining is described. The method involves the steps of quenching underrivatizing, amine stripping and regaining (QUAS-R) of cells, tissue or any biological sample.

The present application claims benefit of Provisional Application 62/374,456, filed Aug. 12, 2016; Provisional Application 62/373,867, filed Aug. 11, 2016; Provisional Application 62/303,243, filed Mar. 3, 2016 and Provisional Application 62/275,949, filed Jan. 7, 2016; all of said applications being expressly incorporated herein.

### FIELD OF THE INVENTION

Formalin fixed paraffin embedded tissue, circulating blood cells, or any mounted biological sample can be stained with fluorescent markers for specific identification and classification. Prior to the present invention, however, staining was limited to 1-5 fluorescent markers. For example, Circulating Tumor Cells (CTCs) are cancer cells that shed from primary/metastatic solid tumors and are found in the circulatory system. For many years whole peripheral blood has been used to isolate CTCs from cancer patients for use as a prognostic indicator of advanced disease. Currently, the only clinically validated prognostic assay isolates CTCs based on antibody mediated capture and identifies CTCs based on three cellular fluorescent markers. This FDA approved assay (CellSearch® CTC Test) captures CTCs from blood using ferrofluid nanoparticles conjugated with antibodies against the epithelial cell adhesion molecule (EpCAM). Captured cells are then identified using the fluorescent markers, DAPI (to stain nuclei and identify an object as a cell), cytokeratin (CK) (to identify the cell as epithelial), and CD45 (to exclude white blood cells).

A plethora of alternative blood cell isolation methods have been introduced. These assays expand the analysis beyond capturing CTCs, to include circulating cancer associated macrophage-like cells (CAMLs), circulating endothelial cells (CECs), epithelial mesenchymal transition (EMT) cells, circulating fibroblasts (CFs), etc. Simple analyte enumeration of CTCs by means of identification and subtyping of the cells is inadequate for many applications. Regardless of the isolation platform, fluorescence detection is the usual means of cellula identification and was previously limited to 4-5 total fluors per cell. This limits fluorescence based cell characterization to the three aforementioned identification biomarkers and 1-2 additional subtyping biomarkers. Clinically and biologically this limits researchers to superficial proteomic identification of cells, while the need to truly interrogate relevant cellular phenotypes requires multiple subtyping markers. Researchers tried to overcome this limitation by collecting cells using multiple biological samples from the same patient, for example collecting many tubes of blood and analyzing cells from each tube for different markers. Cells, however, have enormous phenotypic heterogeneity which makes the staining of individual cells from different blood collection tubes incomparable. Similarly, more than one tissue biopsy slide was needed to analyze more than five markers. A rapid and simple method of using multiple markers to analyze same samples to enhance the diagnosis and treatment would provide enormous clinical benefits.

Identification and classification of cells in diseases is complicated, as different subgroups of cells upregulate and/or down regulate phenotypes in relation to disease progression, disease spread, and in response to disease treatments. For example, the ability of cancer cells to transition to different states, such as the epithelial to mesenchymal transitions, or alter expression of inflammatory immune checkpoints are examples of the active state of tumors changing dynamically in real time as the cancer progresses or responds to treatment. These changes occur in many disorders and thus, circulating cells (e.g. CTCs, CAMLs, CECs, EMTs, etc.) are uniquely suitable as a possible representative surrogate biomarkers for tracking disease progression in real time.

One example of the need for fluorescence staining for more than 4-5 markers is CTCs undergoing EMT. This phenomenon is common in cancer patient blood and has been implicated as a primary cellular component in metastatic spread. Unfortunately, EMT has no universally accepted positive set of biomarkers and is generally described by the down regulation of epithelial proteins, e.g., EpCAM and CK, and the upregulation of mesenchymal stem cell proteins, e.g. vimentin and CD34. EMT is currently a topic of great interest, however, because of the limited proteomic analysis from the limited free fluorescent channels, EMT subtyping is typically screened using non-proteomic methods, e.g., mRNA expression or DNA analysis.

In fluorescence based staining of biological samples, borohydride derivatives (e.g. cyanoborohydride and lithium borohydride) are staple reagents used to reduce background autofluorescence without harming proteomic/genomic markers. Interestingly, while borohydride derivatives were previously used to darken fluorescence in biopsied microsections, it was not used to completely remove specific fluorescent dye signals. Borohydride (BH₄) is a mild and selective carbonyl reducing agent that is commonly used in organic chemistry to reduce ketones and aldehydes to alcohols, and/or imines to secondary amines, without reducing amide or carboxy acid functional groups. In microscopy, BH₄ derivatives are often used to quench the autofluoreseence of formaldehyde, and glutaraldehyde in fixed biological samples. Formaldehyde, glutaraldehyde, and many other fixatives becomes autofluorescent when they react with biological samples, such as tissue, cells, proteins, etc. The autofluorescence is caused by accumulation of carbonylated and Schiff-base compounds on the sample A secondary benefit of adding BH₄ derivatives to fixed biological samples is the ability to reduce free aldehyde groups (e.g. aldehyde blocking), which further minimizes nonspecific binding of the histochemical reagents.

Surprisingly, the present invention found that biological samples can be sequentially stained with at least 25 different fluorescent markers, and visualized for accurate cytological assessment, much like classical cancer histopathological subtyping. The present invention provides platforms wherein a biological cell sample is fixed in site, and sequentially restained with fluorescence markers. Following the first round of staining with a panel of 4-5 fluorescent dyes, the cells are imaged, positioned, marked and archived. Marking the cells allows the user to relocate the identical cell after each step in the quenching process. The present invention consists of a method and reagents to stain for more than 4-5 markers involves the steps of Quenching, Underivatizing, Amine Stripping, and Restaining (QUAS-R) of cells. In one embodiment, cells are isolated on a filter, such as CellSieve™ microfilter (Creatv MicroTech). Importantly, despite the complete quenching with borohydride, the technique preserves epitope integrity. The QUAS-R technique was tested on pancreatic cancer patient samples initially stained for DAPI and CTC markers (CK and EpCAM) and CD45 white blood cell marker. The goal is to re-evaluate the cells for mesenchymal stem cell markers (CD34 and vimentin), motility markers (CXCR4 and vimentin), and inflammatory markers (PD-L1 and PD1). The present invention can be used to sequentially analyze, subtype and track these nine distinct phenotypic cancer markers in addition to DAPI on the same cell samples.

Cell samples can be stained with 4-5 fluorescent markers at one time and imaged. Restaining up to >5 times has been demonstrated. Thus, >25 different markers can be evaluated on the same cell.

The limitation of number of time sample can be restained is dependent on the need and the mounting of the sample to prevent cell loss.

The present invention can be applied to any mounted biological sample (e.g., cells collected from blood, tissue biopsy, cells infected with viruses or bacteria, cells collected from urine, cells collected from spinal fluids, cells collected from other body fluids, tissue removed after surgery) and fixed on any mountable substrate (e.g., glass, polymer, metal,)

### BRIEF DESCRIPTION OF THE FIGURES

FIG. 1. Fluorescence quenching of fluorescent markers on MDA-MB-231 cells FIG. 1a. before and FIG. 1b after 1.5 hours in borohydride solution. Length of scale bar is 10 μm.

FIG. 2. Signal intensity of cells versus background for each fluorescent marker. FIG. 2a depicts the average intensity of marker signals. FIG. 2b depicts the background signal. Size of image is 45 μm×45 μm.

FIG. 3. depicts fluorescence quenching of a cytokeratin positive cell at time 0, 30 min, 60 min and 90 min after addition of borohydride. Size of image is 45 μm'45 μm.

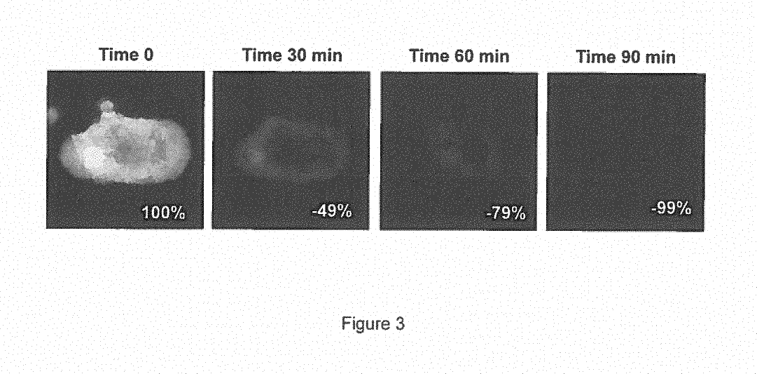

FIG. 4. NADA-MB-231 cells using two QUAS-R rounds. FIG. 4a shows initial staining for cytokeratin, EpCAM and CD45. FIG. 4b shows cells restained for CD14, CXCR4 and vimentin markers after first QUAS-R. FIG. 4c shows cells restained with FD-L1, CD34, and PD1 markers after second QUAS-R. Size of image is 45 μm×45 μm.

FIG. 5. Experimental design and representative examples of the percent change of signal intensity when a marker stain was used on the first round, second round or third round of staining. FIG. 5a shows representative method of staining cells using three QUAS-R rounds. FIG. 5b shows representative signal intensities show no degradation regardless of the stain order.

FIG. 6. Graphs of the overall cellular signal intensity and the changes in nine cellular markers on five different cell lines (HUVEC, MDA-MB-231, A2058, LNCaP, MCF-7) subjected to 2 rounds of QUAS-R. None of the surface receptors nor intracellular markers degraded following the 3 rounds of QUAS-R.

FIG. 7. HUVEC endothelial cells stained with DAPI, anti-CD14 and anti-CD34.

FIG. 8. FIG. 8a shows.EpCAM on MDA-MB-231. White arrow point to high expressing EpCAM cells while gray arrow point to low expressing cells. FIG. 8b is an enlargement of EpCAM stain from Example 2.

FIG. 9. Patient derived EMTs following QUAS-R demonstrates cell subtyping and drug screening targets. FIG. 9a shows initial staining for CK, EpCAM and CD45. FIG. 9b shows restaining far CD14, CXCR4, and vimentin after a first QUAS-R round. FIG. 9c shows restaining for PD-L1, CD34 and PD1 after a second QUAS-R round. FIG. 9d is the heat map of different markers depicting the percentages of marker expressed as dark grey (100%) and white (0%). The grey shades are the percentage from 100% to 0% positive expression. VM=vimentin, CK=cytokeratin. Size of image is 75 μm×75 μm.

FIG. 10. Depicts A2058 cells following two rounds of QUAS-R and varying the staining order. FIG. 10a shows initially staining with CD14, CXCR4 and vimentin FIG. 10b shows the same A2058 cells quenched by QUAS-R and stained with cytokeratin, EpCAM and CD45. FIG. 10c shows the same A2058 were quenched again by QUAS-R and stained with PD-L1, CD34, and PD1.

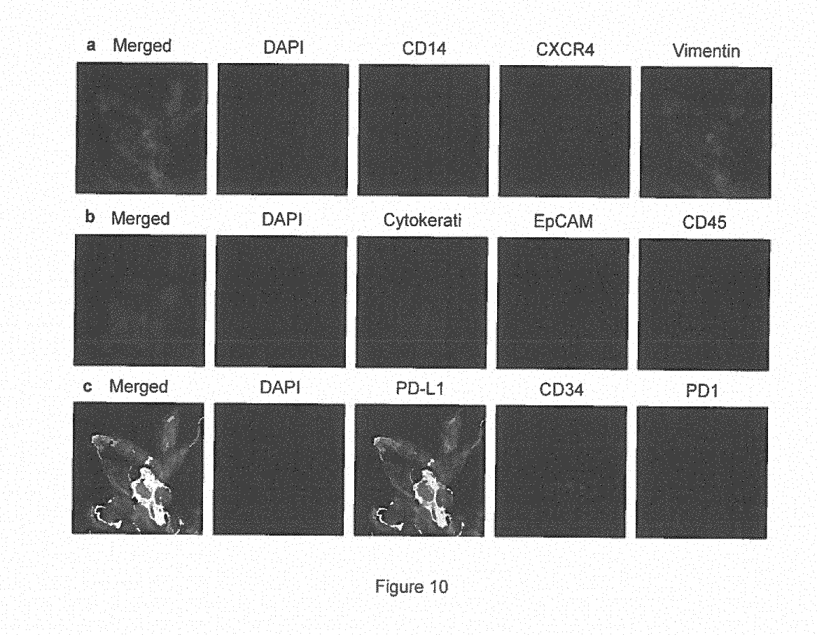

FIG. 11. Depicts the staining of LNCaP cells using two rounds of QUAS-R. FIG. 11a.shows initial staining for cytokeratin, EpCAM and CD45. FIG. 11b. shows restaining for CD14, CXCR4, and vimentin after a first QUAS-R round. FIG. 11c.shows restaining for PD-L1, CD34 and PD1 after a second QUAS-R round.

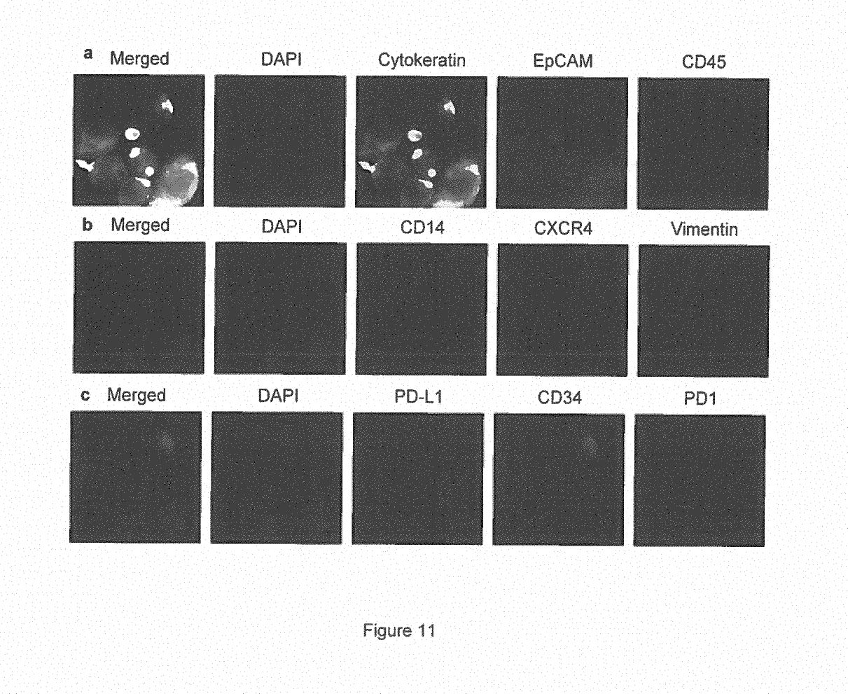

FIG. 12. Depicts the staining of HUVEC cell line using two rounds of QUAS-R. FIG. 12a shows initial staining for CD14, CXCR4 and vimentin. FIG. 12b shows restaining for cytokeratin, EpCAM and CD45 after a first QUAS-R round. FIG. 12c shows restaining for PD-L1, CD34 and PD1 after a second. QUAS-R round.

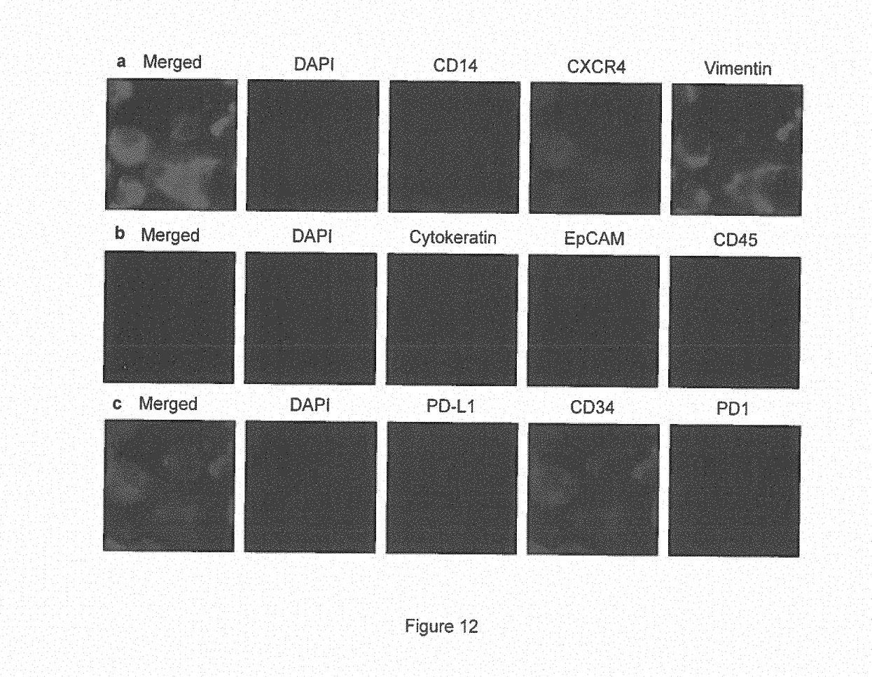

FIG. 13. Depicts the staining of MCF-7 cells using two rounds of QUAS-R. FIG. 13a shows initial staining for PD-L1, CD34 and PD1. FIG. 13b shows restaining for cytokeratin, EpCAM and CD45 after a first QUAS-R round. FIG. 13c shows restaining for CD14, CXCR4 and vimentin after a second QUAS-R round.

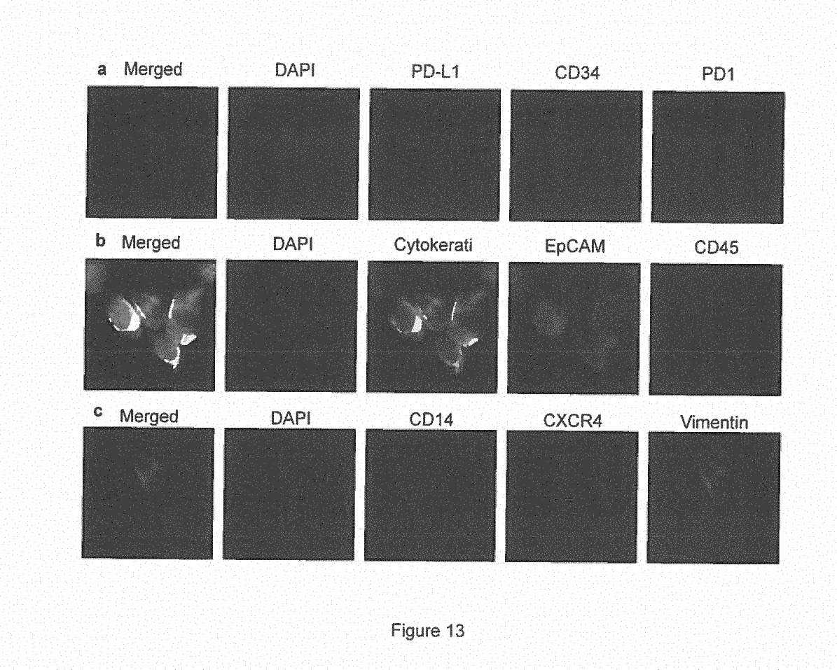

FIG. 14. Heat map of fluorescent marker percentages in five model cell lines. The percentages are expressed as dark grey (100%) and white (0%). The grey shades are the percentage from 100% to 0% positive expression.

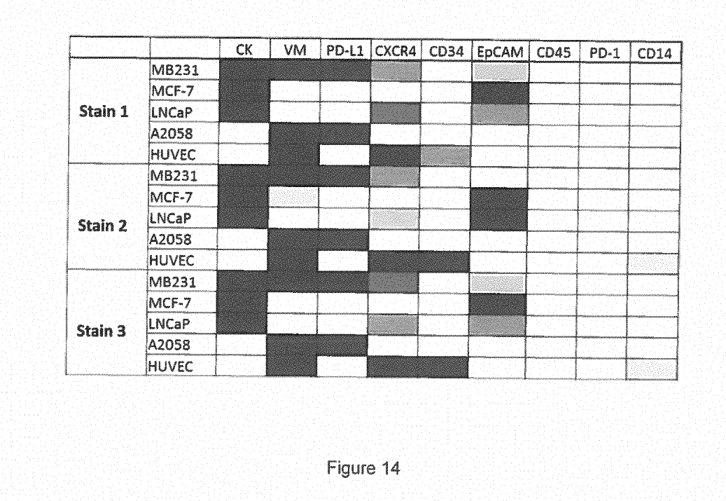

FIG. 15. Heat map of FIG. 9d showing raw percentages. A total of 764 EMTs with a median of 10 cells per sample were measured for the presence of nine markers.

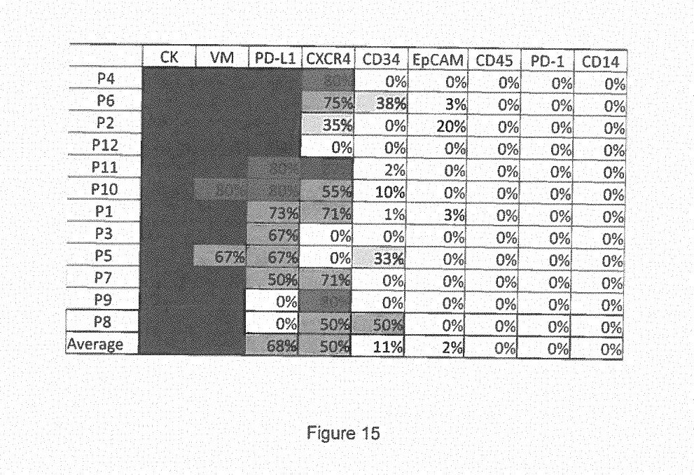

### SUMMARY OF THE INVENTION

The specific embodiments described herein are only representative of the principles of the invention and are not intended to be limited to the disclosed embodiments

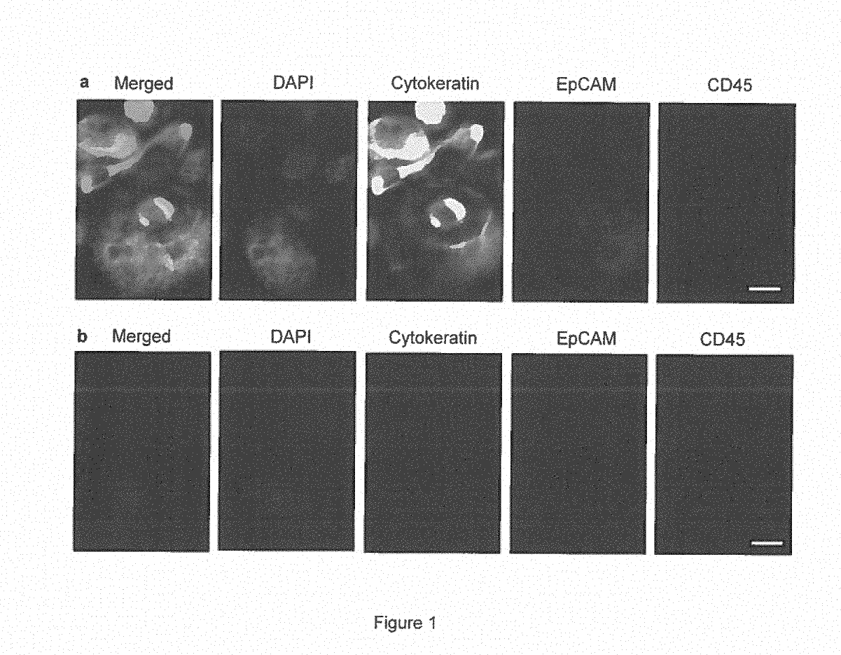

It was discovered that the QUAS-R technique could be employed to rapidly and sequentially stain cell lines with at least twenty five different fluorescent markers in addition to DAPI. In one embodiment, MDA-MB-231 cells, were fixed on a CellSieve® filter and stained using the CTC marker panel: DAPI (blue), CK (green), EpCAM (red) and CD45 (violet). FIG. 1a. The cells were quenched using a borohydride solution for 1.5 hours. 100% of the green of CK, red of EpCAM, violet of CD45, and majority of blue of DAPI, fluorescent signal on the MDA-MB-231 cells was removed. FIG. 1b. The exposure time used for the image acquisition on the microscope is the same before and after quenching.

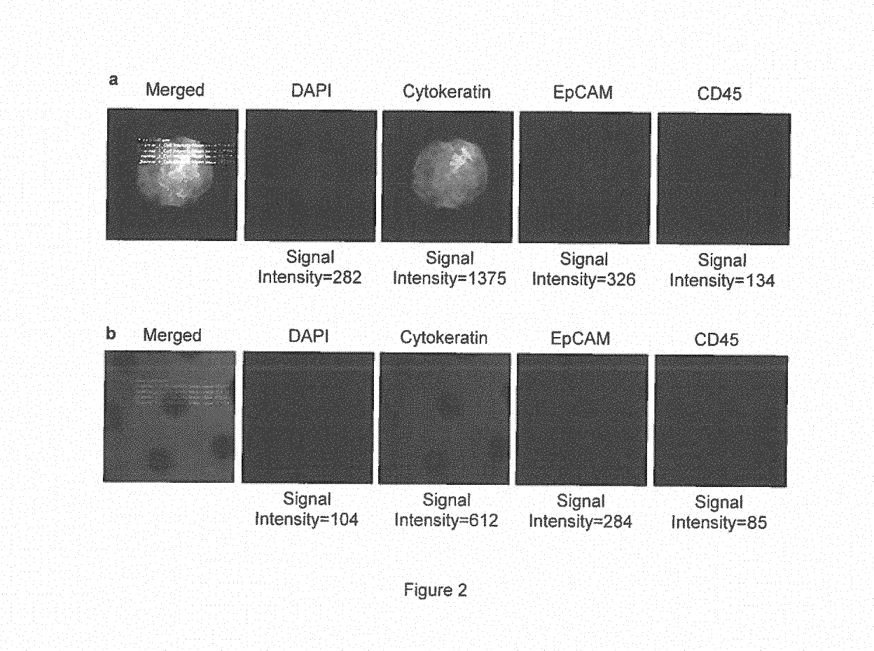

The signal intensity of the cells was compared to the background. FIG. 2. FIG. 2a shows the image of an MDA-MB-231 cell in each fluorescent channel. The average intensity of the signals was measured using Zen2011 Blue software. The signal of each fluorescent channel was compared to an area on the filter without cell, to determine the background. FIG. 2b. The overall stain intensity of each cell was calculated by subtracting the background signal from the cell signal.

In a second embodiment the quenching technique was performed on patient sample stained with FITC tagged cytokeratin antibody. The cell was imaged and the signal measured before addition of Borohydride solution at time 0. Borohydride solution was added and the cell was imaged after 30 min, 60 min and 90 min. After 90 min, 99% of the original fluorescence was quenched.

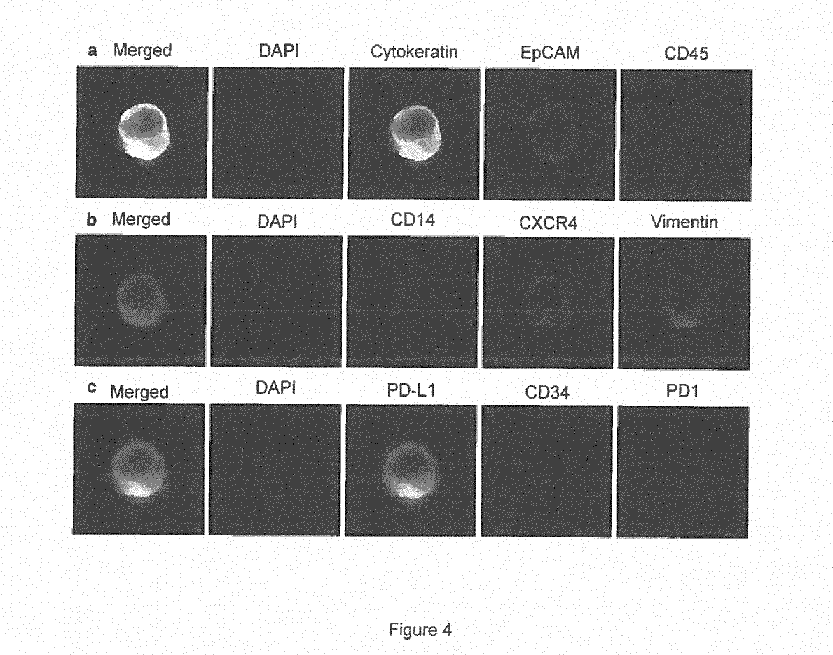

In other embodiments, blood based biopsies (BBBs) or other biological samples were employed to analyze multiple types of cell types (e.g. CTCs, CAMLs, endothelial cells, fibroblasts, etc.) with many different markers. Epitope integrity was maintain when QUAS-R was performed on five cell lines (MDA-MB-231, MCF-7, LNCaP, A2058, and HUVEC) of breast, endothelial, prostate and melanoma origin. FIGS. 4, 5 and 6.

Since immunahistochernistry (IHC) is used in cancer subtyping for both intracellular and extracellular epitopes, nine markers with a broad range of cellular localization both intracellularly (cytokeratin and vimentin) and extracellularly (EpCAM, CD45, CD31, CD34, PD-L1, CXCR4 and CD14) are exemplified herein. In one embodiment MDA-MB-231 cells were fixed on a filter and stained with the CTC stain cytokeratin, EpCAM and CD45. FIG. 4a. The cells were quenched by QUAS-R method and stained with CD14, CXCR4 and vimentin. FIG. 4b. The cells were quenched again by QUAS-R method and stained with PD-L1, CD34, and PD1 FIG. 4c.

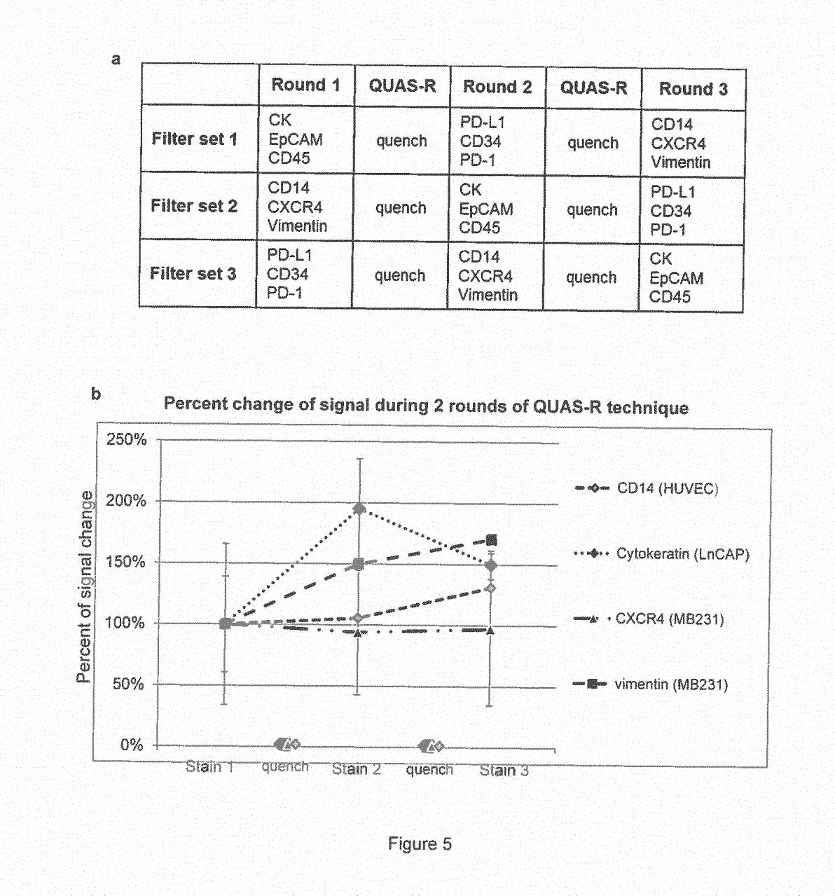

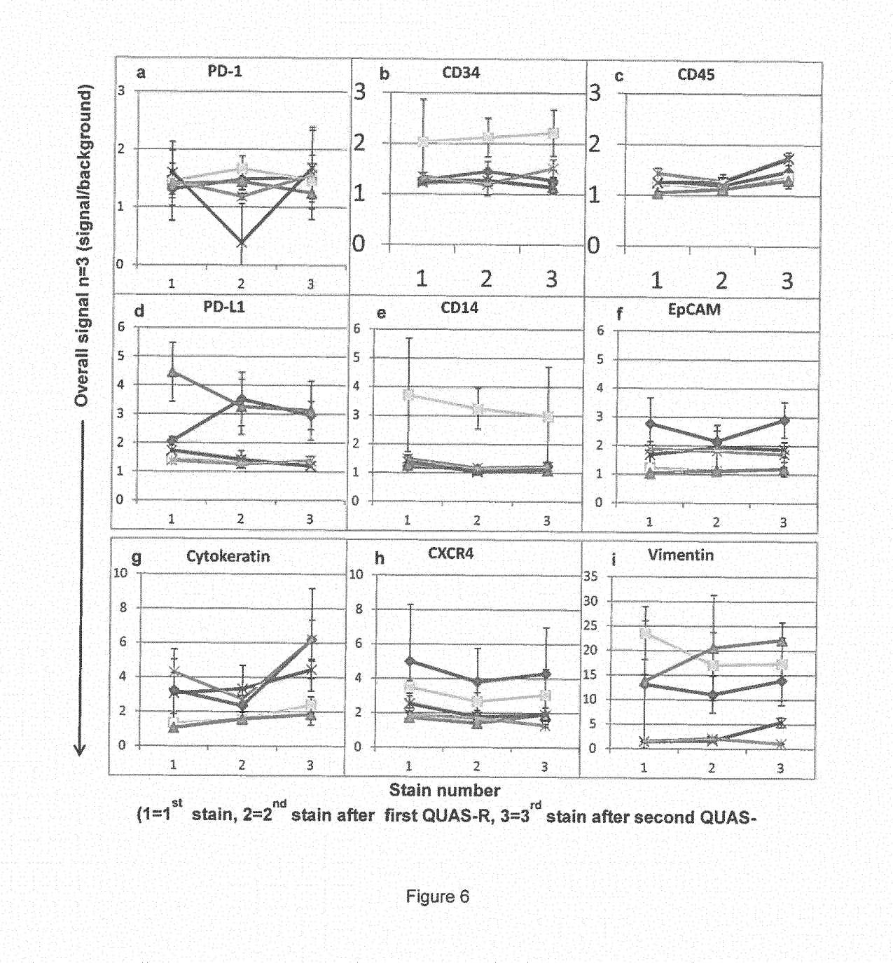

In one embodiment, five cell lines (A2058, LNCaP, MDA-MB-231, MCF-7 and HUVECs) were fixed on filters and stained with nine different markers in varying order. FIG. 5. One filter set, each with one of the five cell lines was stained using the CTC markers (antibody against CK conjugated to FITC (CK-FITC), antibody against EpCAM conjugated to PE (EpCAM-PE) and antibody against CD45 conjugated to Cy5 (CD45-Cy5)). On a second set of filters, the five cell lines were stained with a panel consisting of PD-L1-FITC, CD34-PE and PD1-Dylight 650. On a third set of filters, the five cell lines were stained with a panel of CD14-FITC, CXCR4-PE and vimentin-efluor660. FIG. 5a. The background signal was determined and the intensity of each marker (each marker averaged from 10 cells) was normalized. Then, QUAS-R was performed on all filters and restained each set with a second marker panel. FIG. 5a. The intensity of each marker was measured for all cell types and normalized to background. FIG. 6. A second QUAS-R was performed and each filter set was restained with a third marker panel. FIG. 5a. Signal intensities showed no degradation regardless of order in which the stains were applied. A repeated measure ANOVA showed no significant difference in signal intensity between the three sequential stainings [vimentin MDA-MB-231 cytokeratin LNCaP (p=0.291), CD14 HUVEC (p=0.499), and CXCR4 MDA-MB-231 (p=0.857)].

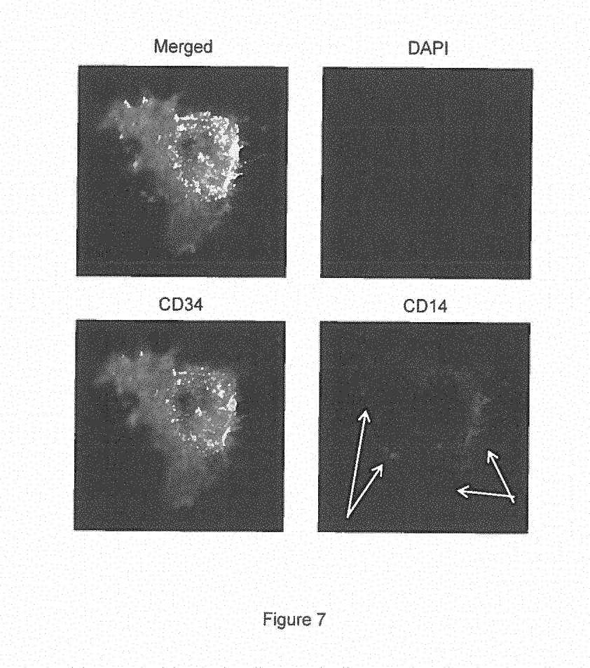

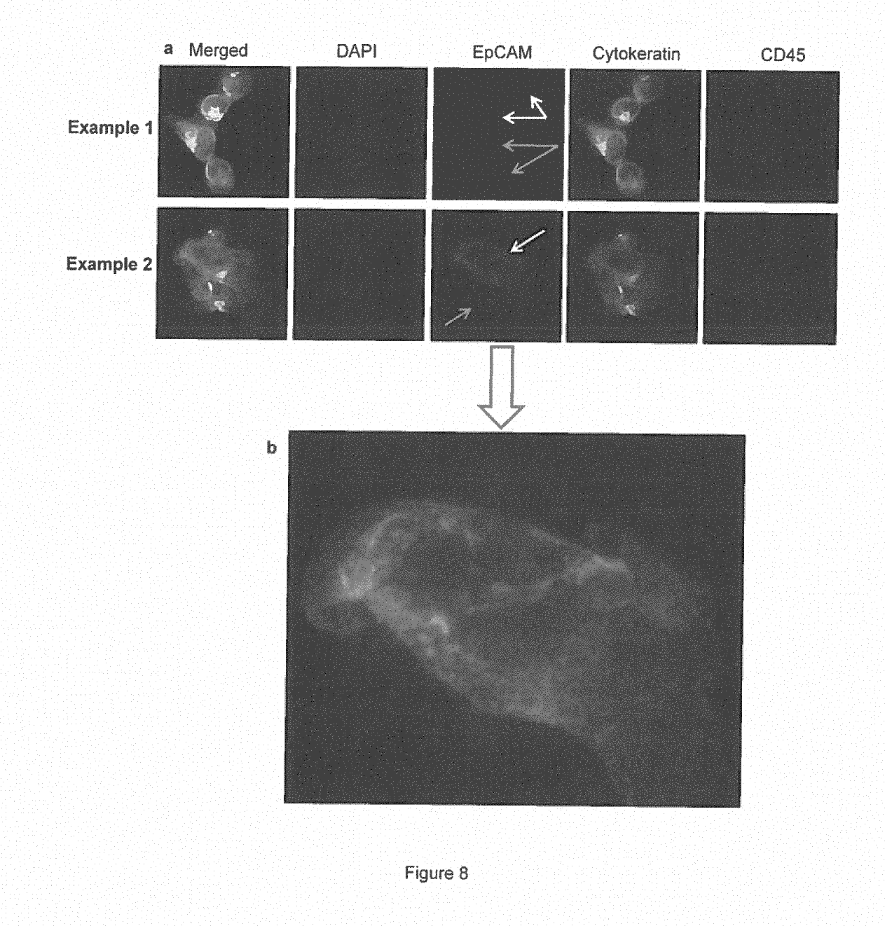

The overall cellular signal intensity and the changes in the nine cellular markers on the five cell lines subjected to QUAS-R is shown in FIG. 6. Neither the surface receptors nor intracellular markers were degraded in any of the QUAS-R rounds. The cells were imaged using a fluorescent microscope with Zen Blue imaging software. A single average pixel intensity of each cell is given by the software, from a range of 0-44096. A signal average pixel intensity of the local background for each image is given by the software, range 0-4096. A signal is considered positive if a cell pixel has an intensity of at least two times the local background pixel intensity. A high positive signal is typically considered a cell intensity of four times the background. The scale can change slightly depending on the fluorescent dyes,, filter cubes, microscopes and exposure duration. FIG. 6a shows that PD-1 was negative in all the cell lines. FIG. 6b shows that CD34 is weakly positive in the HUVEC cell line and as such appears as a low overall signal. FIG. 6c shows that CD45 was negative in all the cell lines. FIG. 6d shows that PD-L1 is variable as indicated by the large standard deviation (SD), but is largely expressed in A2058 and MDA-MB-231 cells. FIG. 6e shows that CD14 was only positive in HUVEC and highly variable as it is only expressed in the protrusions on the cells. Further, the CD14 signal was localized to the peripheral protrusions of a subset of HUVECs (FIG. 7) causing the interior of the cell to have low/no signal. FIG. 6f shows that EpCAM is present as a variable expressing surface marker on LNCaP, MCF-7 and MDA-MB-231. A low overall signal is caused by localization of the marker. EpCAM appeared low in expression and high SD, as the signal was highly heterogenous between cells, and the receptors were aggregated on the cell surface, causing cells to have areas of high and low signals. FIG. 8. FIG. 6g shows that cytokeratin is present as intracellular filaments in LNCaP, MCF-7 and MDA-MB-231 cells. FIG. 6h shows that CXCR4 is a highly variable surface marker found largely in MDA-MB-231 and HUVEC cells. CXCR4 was correctly localized to the MDA-MB-231 cells, albeit with a large SD from the highly varying cell populations. FIG. 6i shows vimentin is present as intracellular filament in HUVECs, MDA-MB-231 and A2058 cells.

PD-L1 and vimentin all showed intense staining in the correct cell lines (MDA-MB-231:high vimentin/high PD-L1, A2058: high vimentin/high PD-L1, and HUVEC:high vimentin) and low/no staining in the correct cell lines (HUVEC:no PD-L1, LNCaP: no vimentin/low PD-L1, and MCF-7:no vimentin/low PD-L1), while cytokeratin stained MCF-7 MDA-MB-231 and LNCapP strongly. Importantly, none of the biomarkers diminished in intensity between the two restains and appeared with appropriate staining intensity in each of the proper cell lines. FIG. 6. The possible exception was of PD-L1, which did appear to slope down during the third staining, but was within the SD. Taken together, these experiments demonstrate that QUAS-R provides full quenching of fluorescently labeled cells and enables restaining of biological specimens, without negatively affecting the quality of their epitopes.

Another embodiment variability of EpCAM expression was exhibited in MDA-MB-231 cells. FIG. 8. In FIG. 8a the white arrow points to high expressing EpCAM cells while the gray arrow points to low expressing cells. FIG. 8b shows a zoomed image of the EpCAM signal from second panel. EpCAM is diffuse and punctate throughout the cell, causing the overall cell signal intensity to appear numerically low.

### Patient Samples

It has been shown that a standard CTC fluorescent staining panel can identify, quantify and score the same clinically prognostic CTCs as the FDA approved CellSearch® System However, the present invention enables the identification of numerous additional subtypes of EMTs, including cells with downregulated cytokeratin signal and no EpCAM signal. While the downregulation of epithelial markers is a hallmark of EMTs in cancer, additional confirmation of upregulated mesenchymal markers is critical to properly profile their stem cell and motility characteristics.

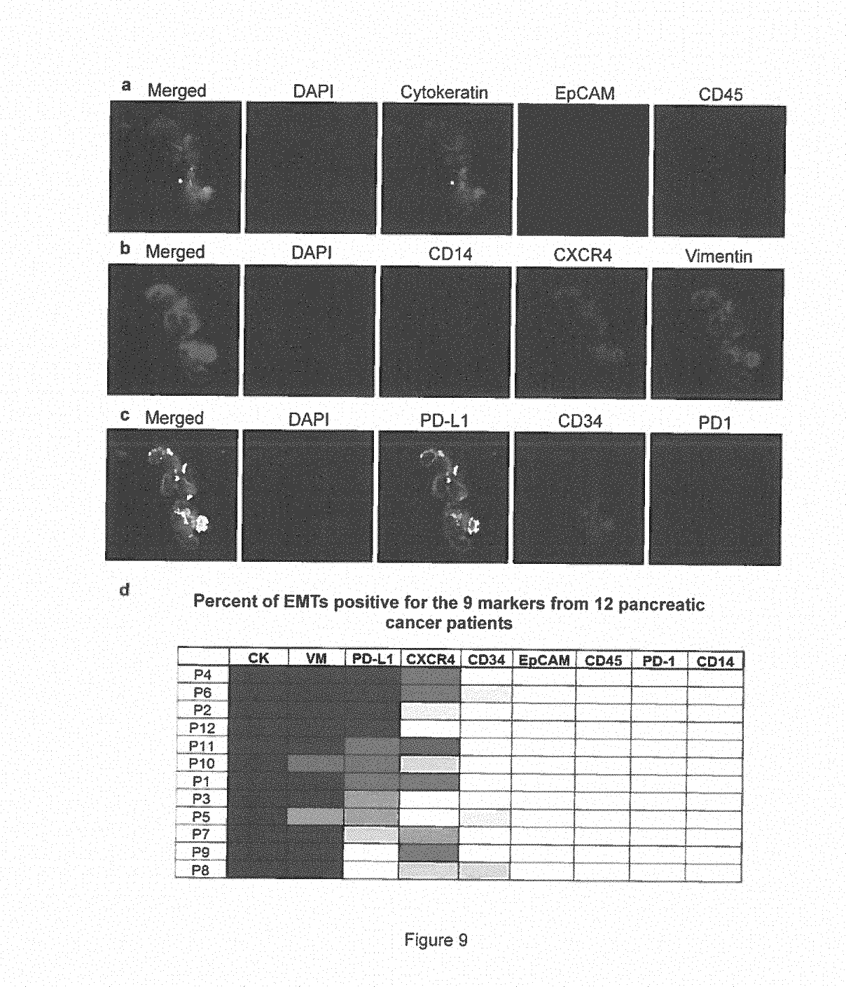

In one embodiment, blood samples from pancreatic cancer patients were filtered and the cells collected on the filter were stained for EMT marker panel (CK and vimentin) and CD45, and imaged. EMTs were found in 78% of pancreatic samples, regardless of stage, while none of the control samples tested positive for EMTs, CAMLs, or CTCs. The cancer patient samples had a total of 764 EMTs with a median of ten cells per sample. QUAS-R was performed on all cancer samples to detect the presence of nine markers. The expression profiles for CK, EpCAM and CD45 from a stage IV pancreatic sample is shown in FIG. 9a. The cells were restained and reimaged with anti-CD14, anti-CXCR4 and anti-vimentin. FIG. 9b. After imaging all cells, QUAS-R was performed a second time and the sample was restained with anti-PDL-1, anti-CD34 and anti-PD-1. FIG. 9c. The presence of each marker is represented as a heat map of percent EMTs positive for each stain. FIG. 9d. The heat map shows the percentages expressed as dark grey (100%) and white (0%). The grey shades are the percentage from 100% to 0% positive expression.

Not surprisingly, all EMTs were positive for CK and negative for CD45, CD14 and PD-1 were also negative in all EMTs, as these are markers for macrophages and activated T-cells, respectively. EpCAM was very uncommon in this cell type, occurring in only 2% of the cell population. FIG. 9d. The absence of EpCAM in EMTs is a well-recognized part of the EMT process. Vimentin was the next most prevalent marker and was found in all but three cells, or 99% of all cells. This is not surprising as the EMT process is reported to downregulate cytokeratin and upregulate vimentin. This process has been shown to increase a cell's mesenchymal phenotypes and allow cells improved motility. The prevalence of CXCR4 in some patients indicates that these mesenchymal cells are migratory. Expression of PD-L1 and CXCR4 was highly heterogeneous between patients, ranging from 100% to 0% positivity for PD-L1 and 90% to 0% positivity for CXCR4, Additionally CD34 was rarely found, with only five cells in three patients being weakly positive.

Using a standard CTC fluorescent stain, EMTs were identified, quantified and scored according to the presence of cytokeratin, EpCAM and CD45. The identification of EMTs from patient samples of circulating cells shows the power of the QUAS-R technique for diagnostic and treatment purposes.

These experiments demonstrate that cells in biological samples can be reimaged and subtyped with multiple immuno-targets, and these markers can be quantified and scored. Unlike a multi-panel IHC testing in classical formalin fixed paraffin embedded (FFPE), a biopsy which uses multiple different slices of biological samples, the QUAS-R method allows the same biological sample to be restained with multiple biomarkers.

### DETAILED DESCRIPTION OF THE INVENTION

Obtaining biological samples can be difficult and clinically important biomarkers are at best, superficially identified when only 2-3 positive fluorescent markers and one negative fluorescent marker are used, limiting additional classification markers. The current practice of using classical tissue biopsies to test for clinical information about the underlying biology require testing different slices from the same biological samples (eg., FFPE slices). For example, in cancer pathology, IHC is the standard biological assay accomplished using thin slices (5-10 μm) cut from a large FFPE biopsied tissue section. However, FFPE slices are not identical and thus each stain is administered to different cells. Staining multiple slices each having different populations of cells, with a variety of subtyping markers does not provide consistent results necessary for diagnosis and treatment. Blood based biopsies (BBBs), on the other hand, typically only isolate a few relevant cells types so the majority of samples (74%) contain less than two of the crucial diagnostic cell types (e.g., CTCs, CECs, etc.) (0-81 CTCs/7.5 ml sample). In both of these circumstances there is a need to both identify the type of cells and to proteomically subtype them for clinically relevant markers.

EMTs (cells with low cytokeratin and low/no EpCAM) are commonly isolated from breast, prostate, lung and pancreatic cancers. However, additional information is often needed about the cancer of the patient requiring the need to stain for additional markers. As multiple biomarker interrogation is necessary to properly identify EMTs, and there is no single biomarker panel for EMTs, testing these cells with a panel of EMT indicative biomarkers is required for accurate diagnosis. In the past, multiple tubes of blood were required to obtain the EMTs for staining for more than 4-5 markers.

The technical difficulty in profiling biological samples cannot be underestimated. For example, tissue biopsies are used to detect the presence of cancer cells and perform companion diagnostics for drug targets. For immunotherapy, additional testing with the appropriate markers is required to characterize any tumors and analyzed the tumor microenvironment, for presence of T-cells and macrophages, To accurately identify the tumor cells, stain for the drug targets, and evaluate the tumor microenvironment requires the use of multiple slices of FFPE samples numerous biomarker stains. The number of biopsied tissue samples available for testing is often limited and does not allow the use of more than 4-5 biomarker stains. Sequential multi-panel restaining of FFPE tissue, or any biological sample, using 9-25 clinically applicable biomarkers provides greater information on the cell subtypes present.

Another embodiment involves the analysis of cancer associated cells in patients' blood. One technical difficulty in profiling CTCs in blood samples is their rarity. Since blood samples from cancer patients usually contain ˜1 CTC per 10⁹ blood cells, in the past this limited the ability to perform detailed cell profiling. CTCs must be identified fluorescently with DAPI, cytokeratin to identify CTCs and CD45 to exclude white blood cells; leaving only 1-2 channels available for proteomic subtyping. Additional staining of CTCs beyond the standard markers (DAN, EpCAM, CK and CD45) have been reported, but this staining has mostly been limited to 1-2 additional markers. Examining CTCs isolated from duplicate or multiple samples from the same patient could provide some degree of multiplexing, however, this option did not produce consistent and reliable results since tumor cells in blood are rare, very heterogeneous and unevenly distributed. The clustering of CTCS in blood samples results in only 39% of the CTCs being enumerated, which is on par with the clustering of CTCs observed in highly heterogeneic pancreatic cancer biopsies. As shown in FIG. 9, the variation in intensity between pancreatic cancer cells can be visualized in cytokeratin, EpCAM, CXCR4, vimentin, PD-L1 and CD34. The QUAS-R technique enables the expansion of proteomic cell subtyping of blood samples with the use of 9-25 unrelated fluorescent antibodies in a simple inexpensive quenching and restaining method.

Many important cell types, other than CTCs, are present in the blood of cancer patients. For example, CAMLs are circulating tumor derived cells that have many potential clinical applications including early cancer detection. The markers that consistently appear on CAMLs include, among others, CD14, CD34, CD146, CD11c. Circulating EMTs can be consistently identified by the fluorescent stain vimentin. Thus, the use of QUAS-R to stain with a large panel of various markers is essential for improved identification of all the cells of interest present in a patient's blood.

Recently, it was shown that partial BK₄ quenching on photoactivatable fluorophores does not alter the quenched proteins and is compatible with high resolution microscopy. The borohydride attributes were described using any and all derivatives of borohydride (e.g., sodium borohydride, lithium borohydride, cyanoborohydride, etc) and described as well suited for temporary reduction of fluorescent signal from biological samples without concern for destruction of epitopes. However, the prior art only slightly and temporarily darken the fluorescence, and did not completely remove the fluorescence. The QUAS-R technique involves the use of borohydride for permanent quenching of specific fluors, also without epitope damage. Total IHC specific fluorescence can be removed from bound antibodies without harming the visualization or quantification of IHC epitopes. The borohydride solution also removes residual background fluorescence and is able to unmask any blocked epitopes. At least six separate conjugated fluors (alexafluor488, FITC, efluor 615, efluor 660, PE, APC, and Cyanine5) have been completely darkened using this method as well as any organic dye (e.g., efluor, nanocrystals, etc). The examples describe the use of QUAS-R on BBBs, however, the method is applicable to any biological assay involving a fixed or unfixed sample

The examples demonstrate that EMTs are all CK expressing and mostly vimentin expressing. Of course EMT is a transient process defined by a number molecular and proteomic pathways not all of which have been completely identified. The ability to stain with multiple cell markers enables the elucidation of the underlying biology of CTCs which have begun the EMT process, e.g., low/negative CK and EpCAM, while verifying that the cells are not originally of hematopoietic (CD45) or myeloid (CD14) lineage. The hiornarker panel can now be expanded to include all of the known biological cell types and marker types. For EMTs, these panels can include other markers such as N-Cadherin, TWIST, SNAIL, ZEB1. The QUAS-R method uses a combination of manual identification and AxioVision's Mark and Find module. However, it can be adapted to a folly automated system to streamline the process. Automated or manual, the ability to screen biological samples beyond basic identification using multiple identification and classification biomarkers without the need for excess sampling or use of non-matching sample slices greatly enhances the diagnostic accuracy and clinical utilities.

### EXAMPLES

### Example 1

### Healthy and Patient Blood Samples

Twelve whole peripheral blood samples were drawn from patients who were actively undergoing treatment for stage I-IV pancreatic cancer at The Medical College of Wisconsin from 2012 to 2013. Samples were collected in accordance with and approved by the local Institutional Review Board (IRB) at the Medical College of Wisconsin, with the patient signed informed consent. All blood samples were drawn into CellSave preservative tubes™ (˜9 mL, Janssen Diagnostics) and shipped to clinical core laboratory for processing. Results and patient identification from institutions were not shared or communicated until completion of study. All patient samples were first labeled with a standard antibody mixture for staining epithelial cells consisting of FITC labeled-anti-cytokeratin 8, 18, 19; r-Phycoerythrin (PE) labeled anti-EpCAM; and Cyanine5 labeled anti-CD45.

Blood samples from anonymous healthy volunteers (n=12) were procured with written and signed informed consent. The informed consent was in accordance with and approved by Western Institutional Review Board. Donor blood was at a blood collection center using standard exclusion criteria, e.g., all samples were considered from normal, healthy individuals. The blood samples were drawn into CellSave preservative tubes and shipped to a clinical core laboratory for processing.

### Example 2

### CTC Staining Procedures Performed on CellSieve™ Filters.

Samples were filtered with a CellSieve™ CTC Enumeration Kit reagents (Creatv MicroTech) using a low-pressure vacuum system which isolates CTCs based on size exclusion, ˜7 micron. Cells were stained and identified by fluorescence using CTC enumeration stains (example 4). A low-pressure system was created using a filter holder assembly with a CellSieve™ filter attached. Peripheral blood (7.5 ml), was diluted in a prefixation buffer and drawn through the filter. The filter was washed, postfixed with CellSieve™ Postfixation buffer and permeabilized using CellSieve™ Premeabilization buffer. The captured cells were stained with an antibody cocktail consisting of FITC-anti-cytokeratin 8, 18, 19, PE-anti-EpCAM and Cy5-anti-CD45 for 1 hour and mounted with Fluoromount-GDAPI (Southern Biotech). An Olympus BX54WI Fluorescent microscope with Carl Zeiss AxioCam was used to image the samples. Exposures were preset at 2 sec (Cyanine5), 2 sec (PE), 100-750 msec (FITC), and 10-50 msec (DAN) for equal signal comparisons between cells. A Zen2011 Blue (Carl Zeiss) with AxioVision Mark and Find module was used to process the images, mark the x/y placement of the cells, and relocate previously imaged cells in a semi-automated manner. Samples were archived and placed in storage at 4° C. for one week to two years.

### Example 3

### Cell Lines.

MCF-7 (HTB-22) and MDA-MB-231 (HTB-26) human breast cancer cell lines; LNCaP (CRL-1740, clone FGC) prostate adenocarcinoma; A2058 (CRL-11147) human skin melanoma cell line; and HUVEC-C (CRL-1730) endothelial cells were procured from ATCC (Manassas, Va.). All cell lines were grown in cell line specific media containing fetal bovine serum (FBS) as recommended by ATCC. Cell lines were maintained in T-75 flasks using prescribed cell culture conditions (5% CO₂, 37° C.) with media changes every 3-4 days, with the exception of the MDA-MB-231 cell lines, which were grown at 37° C. with no added CO₂. Cells were harvested using trypsin-EDTA (ATCC Manassas, Va.), spun at 125×g for 5 min resuspended in PBS containing 1% paraformaldehyde. After incubation, cells were diluted in 10× volume of PBS, centrifuged and resuspended in fresh PBS before being spiked into normal blood and isolated using microfilters within 5 min.

### Example 4

### Additional Marker Panels

Markers are identified by antibodies against the markers. Samples were stained by incubating with fluorescent labeled antibodies for 1 hour at room temperature. Primary CTC panel: FITC labeled-anti-cytokeratin 8, 18, 19; r-Phycoerythrin (PE) labeled anti-EpCAM; and Cyanine5 labeled anti-CD45 (Creator MicroTech), second panel: Alexafltior 488 labeled anti-PDL1 (2.5 ug/mL) and Dylight 650 labeled PD-1 (5 ug/mL) both PD-L1 and PD-1 were gifts from Dr. Steven Lin, MD Anderson Cancer Center, PE labeled CD34 (2.5 ug/ml, clone 4H11) and third panel: FITC labeled anti-CD14 (5 ug/ml, clone 61D3), PE labeled anti-CD184 (5 ug/ml, clone 2B11), efluor660 labeled anti-vimentin (2.5 ug/ml, clone V9).

An Olympus BX54WI Fluorescent microscope with Carl Zeiss AxioCam was used to image the samples. Exposures were preset at 2 sec (Cyanine5 and APC), 2 sec (PE), 1000 msec (FITC and Alexafluor 488), 500 msec (efluor660), and 10-50 msec (DAN) for equal signal comparisons between cells. A Zen2011 Blue (Carl Zeiss) was used to process the images, mark the x/y placement of the cells and relocate previously imaged cells.

### Example 5

### 1. Fluorescent Quenching (QUAS-R) of Cells on Filters

Archived samples were removed from storage one week to two years after initial CTC staining. Samples had been previously stained, imaged and marked prior to the quenching procedure. Slides were soaked in 100 mL 1X PBS for 15 minutes and carefully demounted. Filters were placed into a reaction chamber (Coming) and washed five times with 1 mL 1X PBS,

Quenching: Filters with bound cells were incubated with 1 mg/ml sodium borohydride solution (Fisher Scientific) for one hour at room temp in a chemical hood. The borohydride solution was removed and filters were washed six times with 1 ml 1X PBS.

Underivatizing and Amine Stripping: During aldehyde fixation the polymers in the fixatives react and cross links proteins. As the sample ages the polymers degrade and various polymer derivatives form. Underivatizing is a term I made up to describe the removal of the various polymer derivatives. Aldehyde fixation (like glutaraldehyde or formalin) reacts with amines and proteins causing autofluorescence. Amine stripping washes away the free and reacted amines and the autofluorescence associated with them These steps consist of (a) placing the filters in a clean reaction chamber (Corning) and incubating with 100 mM Tris pH=9.0 for one hour at room temperature to remove borohydride, and (b) removing Tris by washing the filters three times with 1 ml 1X PBS.

Restaining: 1XPBS/20%FBS was added to the chamber to block the cells for 30 minutes. After incubation, the TABS/FBS solution was removed. The next set of antibody stain was added to the chamber for 1 hour at room temp. Following antibody incubation, the filters were washed in 1X PBS/1%Tween and the slide was mounted with Fluoromount-GIDAN (Southern Biotech).

Samples were oriented along the x/y axis and the previously imaged cells were relocated using a fluorescent microscope and software, such as Zen2011 Blue (Carl Zeiss) software. Images and exposures were preset as described above and a Zen2011 Blue (Carl Zeiss) was used to process the images. Following imaging of the fluorescent markers on the cells, QUAS-R procedure was repeated with the next antibody cocktail and reimaged. For time gated experiments involving visualizing fluorescence quenching, filters were placed under a fluorescent microscope (such as Olympus) in a ventilated hood and imaged with the filter remaining in the borohydride solution.

### 2. Fluorescent Quenching (QUAS-R) of Cells or Biopsies Mounted on Glass Slides

Archived biopsy samples were removed from storage one week to two years after initial fluorescent staining. Samples had been previously imaged and marked prior to the quenching procedure. Slides were soaked in 100 ml 1X PBS for 15 minutes. Slides were washed five times with 1 mL 1X PBS. Slides were then coated, or dipped into a Coplin jar containing 1 mg/mL sodium borohydride solution (Fisher Scientific) for 1 hour at room temperature in a chemical hood. The borohydride solution was removed and the slides were washed six times with 1 ml 1X PBS. The slides were placed in a clean Coplin jar and incubated with 100 mM Iris pH=9.0 for one hour at room temperature. The Iris was removed and the slides were washed three times with 1 ml PBS and placed in a Coplin jar with 1XPBS/20%FBS for 30 minutes. Following incubation, the PBS/FBS solution was removed and the next set of antibody stain was added to the biopsy sample for one hour at room temperature. Following antibody incubation, the slides were washed in 1X PBS/1%Tween and the slide mounted with Fluoromount-G/DAPI (Southern Biotech). Samples were oriented along the x/y axis and previously imaged cells were relocated using a fluorescent microscope and software, such as Zen2011 Blue (Carl Zeiss) software. Images and exposures were preset as above and a Zen2011 Blue (Carl Zeiss) was used to process the images. After imaging the fluorescent markers on the cells, QUAS-R procedure can be repeated with the another antibody cocktail and reimaged.

### Example 6

### Sequentially Screening Biomarkers in Cell Lines

Each cell line was individually filtered onto a microfilter and each cell type (n=2) was stained with either antibody panel 1 (CK, EpCAM, and CD45), antibody panel 2 (PD-L1, CD34, and PD-1), or antibody panel 3 (CD14, CXCR4 and vimentin). FIG. 5a. After imaging and marking, each individual filter was quenched by the QUAS-R method, as described above, and then restained with a second antibody set, i.e. filter set 1 was originally stained with CK, EpCAM and CD45 and was then stained with antibody panel 2 (PD-L1, CD34, and PD-1); filter set 2 was originally stained with PD-L1, CD34, and PD-1 and was then stained with antibody panel 3 (CD14, CXCR4, Vimentin): and filter set 3 was originally stained with CD14, CXCR4, and vimentin and was then stained with antibody panel 2 (CK, EpCAM and CD45). AH originally marked cells were found and reimaged.

After imaging all cell lines, QUAS-R was performed a second time on each filter set and cell line. This time filter set 1 was restained with antibody panel 3, filter set 2 was restained with antibody panel 1, and filter set 3 was restained with antibody panel 1. Again, all originally marked cells were found and reimaged.

Although the invention was described with respect to specific embodiments and examples, the concept of the invention can be broadly applied.

Other diseases and disorders have cells and/or components of interest that can be analyzed using the QUAS-R technique. Cells containing active or inactive viral infections, viral components, bacterial infections, bacterial components, and other diseases and disease components, can also be found in blood and tissue. The markers for each disease or disorder will vary and thus require the staining of different biomarkers.

The affinity component is not limited to antibodies as described in the examples. Other common affinity components such as aptamers, lectins, proteins, enzymes, etc., can also be used in the QUAS-R technique.

Cells are also present in a variety of body fluids including, but not limited to, blood, urine, bone marrow, lymphatic tissue, cerebrospinal fluid, amniotic fluid, bile, saliva, sputum, ascites, pleural effusion, cervical vaginal fluid, ovarian cyst fluid, endometrial fluid, uterine lavage fluid, lymphedema. The QUAS-R technique described herein can also be used to screen for different cells and biomarkers in these body fluids.

Any borohydride derivative can be used to quench samples (e.g., sodium borohydride, lithium borohydride, cyanoborohydride, Tetra-n-butylammonium borohydride. Benzyltriethylammonium borohydride, etc) The aforementioned examples of quenching used the derivative sodium borohydride, but other derivatives also work well.

The QUAS-R technique can be used on any biological sample containing cells. Examples of biological samples include, but are not limited to, formalin fixed paraffin embedded (FFPE) tissue, floating cell, blood cells, cancer cells, diseased cells, tissue from different organs, lymphatic cells, hair, skin, bone marrow, etc. The CTCs are only an example of cells used to illustrate the process and not limit its application.

The QUAS-R technique can be also used on samples mounted on substrates. The type of substrates include, but are not limited to, glass, metal, polymer, plastics, paper, fibrous material, etc.
a. The quenching steps above describe the application of cells mounted on a polymer. This process can be used on all materials on which biological samples are mounted.
b. The QUAS-R technique can be performed on cells in solution, not mounted to a substrate.
c. The technique can be performed on FFPE samples mounted on glass slides.

The QUAS-R technique can be used to quench old samples with autofluorescence caused by age. Aging can cause degradation of the fluors. Aging can also cause degradation of the affinity components (e.g. antibodies, aptamers, lectins, proteins, enzymes, etc).
a. This includes fixed or unfixed samples.
b. In addition to quenching the specific fluoresence, aged samples have additional non-specific fluorescence that requires quenching.
c. The non-specific fluorescence is also quenched during the process.
d. previously unstained samples must be quenched for removal of background fluorescence and naturally occurring autofluorescence.
e. Fixed biological sample or aged samples have epitopes that might be blocked by chemical modifications or alterations in tertiary structure. In addition to quenching a secondary effect of the borohydride is its ability to unblock epitopes for restaining.

QUAS-R protocol has been demonstrated for restaining up to 5 times. The limitation of number of time QUAS-R can be performed on a sample is dependent on the need and the mounting of the sample to prevent cell loss.

The type and concentration of the reagents, incubation time, and protocols to implement the steps of QUAS-R will vary depending on the sample type.

1. A method of restaining a biological sample for biomarkers comprising:
a. quenching the fluorescence with a reducing agent;
b. underivatizing and amine stripping; and
c. restaining the sample with one or more additional fluorescent markers.

2. The method of claim 1 wherein the biological sample was previously stained with one or more fluorescent markers.

3. (canceled)

4. (canceled)

5. (canceled)

6. (canceled)

7. (canceled)

8. (canceled)

9. (canceled)

10. (canceled)

11. A method of screening for biomarkers in a biological sample comprising:
a. quenching the fluorescence with a reducing agent;
b. underivatizing and amine stripping; and
c. restaining the sample with one or more additional fluorescent markers.

12. The method of claim 11 wherein the biological sample was previously stained with one or more fluorescent markers.

13. (canceled)

14. (canceled)

15. (canceled)

16. (canceled)

17. The method of claim 16 wherein the biological sample was previously stained with one or more fluorescent markers.

18. (canceled)

19. The method of claim 16 that further comprises:
a. fixing the sample on a surface; and
b. visualizing the biomarkers.

20. The method of claim 16 wherein the reducing agent is a borohydride derivative selected from the group consisting of sodium borohydride, lithium borohydride, cyanoborohydride, tetra-n-butylammonium borohydride, and benzyltriethylammonium borohydride.

21. (canceled)

22. (canceled)

23. (canceled)

24. The method of claim 1 that further comprises:
a. fixing the sample on a surface; and
b. visualizing the biomarkers.

25. The method of claim 24 wherein the reducing agent is a borohydride derivative selected from the group consisting of sodium borohydride, lithium borohydride, cyanoborohydride, tetra-n-butylammonium borohydride, and benzyltriethylammonium borohydride.

26. The method of claim 25 wherein the borohydride is sodium borohydride.

27. The method of claim 33 wherein the biological sample is selected from the group consisting of blood, urine, bone marrow, lymphatic tissue, cerebrospinal fluid, amniotic fluid, bile, saliva, sputum, ascites, pleural effusion, vaginal fluid, ovarian cyst fluid, endometrial fluid, and lymphedema.

28. The method of claim 33 wherein the biological sample is selected from the group consisting essentially of cells, viral components, bacterial components, and disease components.

29. The method of claim 28 wherein the biological sample is a cell is selected from the group consisting of tissue, cancer associated cells in blood, CTCs, EMTs, CAMLs, CECs, blood cells, lymphatic cells, hair cells, skin cells and bone marrow cells.

30. The method of claim 29 wherein the biological sample is a human cancer cell.

31. The method of claim 34 wherein the reducing agent is a borohydride derivative selected from the group consisting of sodium borohydride, lithium borohydride, cyanoborohydride, tetra-n-butylammonium borohydride, and benzyltriethylammonium borohydride.

32. The method of claim 34 that further comprises:
a. fixing the sample on a surface; and
b. visualizing the biomarkers.

33. The method of claim 1 wherein the biological sample was stored for at least one week.

34. The method of claim 11 wherein the biological sample was stored for at least one week.

35. The method of claim 16 wherein the biological sample was stored for at least one week.
